(19) United States
(12) Patent Application Publication
(10) Pub. No.: US 2005/0074831 A1
(43) Pub. Date: Apr. 7, 2005

(54) Interaction of NMDA receptor with protein serine threonine phosphatases

(76) Inventors: Jasna Jerecic, San Francisco, CA (US); Janice Williams, Richmond, CA (US); Jean Bucaria, Bayshore, NY (US); Thorsten Melcher, San Francisco, CA (US)

Correspondence Address: BOZICEVIC, FIELD & FRANCIS LLP, 1900 UNIVERSITY AVENUE, SUITE 200, EAST PALO ALTO, CA, 94303, US

(21) Appl. No.: 10/386,971

(22) Filed: Mar. 11, 2003

Related U.S. Application Data
(60) Provisional application No. 60/365,970, filed on Mar. 19, 2002.

(51) Int. Cl. C12Q 1/44

(52) U.S. Cl. 435/19

(57) ABSTRACT

The present invention relates to the identification of a binding between NMDA receptor (NMDA-R) subunits and a serine/threonine protein phosphatase (PSTP), e.g., PP2A. The present invention provides methods for screening a PSTP agonist or antagonist that modulates NMDA-R signaling. The present invention also provide methods and compositions for treatment of disorders mediated by abnormal NMDA-R signaling.

### FIELD OF THE INVENTION

The invention relates in general to the N-methyl-D-aspartate (NMDA) receptor and its signaling activity. The invention provides methods for identifying agonists and antagonists of NMDA receptor signaling, as well as compositions and methods useful for treating physiologic and pathologic conditions mediated by the NMDA receptor. The invention finds application in the biomedical sciences.

### BACKGROUND OF THE INVENTION

Many eukaryotic cell functions, including signal transduction, cell adhesion, gene transcription, RNA splicing, apoptosis and cell proliferation, are controlled by protein phosphorylation. Protein phosphorylation is in turn regulated by the dynamic relationship between kinases and phosphatases. Three basic types of eukaryotic protein phosphatases have been defined: serine/threonine protein phosphatases (PSTP's), tyrosine protein phosphatases, and dual-specificity phosphatases (DSP's). The DSP's dephosphorylate tyrosine and threonine residues on the same polypeptide substrate.

The PSTP's are further classified into subfamilies by substrate specificity, metal ion dependence and sensitivity to inhibition. Type 1 PSTP's (PP1) are characterized by their inhibition by protein inhibitors 1 and 2. PP1 preferentially dephosphorylates the β-subunit of phosphorylase kinase. Type 2 PSTP's (PP2) are not inhibited by these inhibitors and are further divided into PP2A, PP2B, and PP2C. PP2 dephosphorylates mainly the α-subunit of phosphorylase kinase. In addition to PP1 and PP2, other types of PSTP's have also been identified, including PP3, PP4, PP5, PP6, and PP7 (see, e.g., Cohen, Trends Biochem. Sci. 22: 245-251, 1997; Honkanen et al., J. Biol. Chem. 266:6614-6619, 1991; and Herzig et al., Physiol. Rev. 80:172-210; 2000).

Most PSTP's are multimeric proteins that consist of the catalytic subunit and one or more accessory proteins. The accessory proteins confer substrate specificity, regulate enzyme activity, and control the subcellular localization of the holozyme (Paux et al., Trends Biochem. Sci. 21:312-315, 1996). The catalytic subunits of most PSTP's are encoded by the PPP gene families (Cohen, Trends Biochem. Sci. 22: 245-251, 1997). The PPP family includes PP1, PP2A, PP2B, PP4, PP5, and PP6. In contrast to PP1PP2A, and PP2B, PP2C is monomeric and its catalytic subunit is encoded by the PPM gene (Cohen, supra; and Shenolikar et al., Adv. Second Messenger Phosphoprotein Res. 23: 1-121, 1991).

PP2A can be dimeric or trimeric. Trimeric PP2A contains a catalytic subunit (C), a structural subunit (A), and a regulatory subunit (B). Dimeric PP2A are formed of equimolar A and C subunits. It is not entirely clear whether the native enzyme is dimer or trimer. Two isoforms of PP2A catalytic subunits exist, PP2Aα and PP2Aβ. They have been isolated from various species (see, e.g., Herzig et al., Physiol. Rev. 80:172-210, 2000). The B-subunit family comprises B-α, B-β, and B-γ. The A, B-α and C subunits are expressed in many tissues, while B-β and B-γ subunits are detectable only in brain and components of the brain PP2A. B-α and B-β are mainly cytosolic, and B-gamma is enriched in the cytoskeletal fraction. In addition, the A, C, B-α subunits are expressed at constant levels. By contrast, B-β expression decreases after birth while B-γ expression increases after birth.

In the majority of mammalian excitatory synapses, glutamate (Glu) mediates rapid chemical neurotransmission by binding to three distinct types of glutamate receptors on the surfaces of brain neurons. Although cellular responses mediated by glutamate receptors are normally triggered by exactly the same excitatory amino acid (EAA) neurotransmitters in the brain (e.g., glutamate or aspartate), the different subtypes of glutamate receptors have different patterns of distribution in the brain, and different cellular signal transduction. One major class of glutamate receptors is referred to as N-methyl-D-aspartate receptors (NMDA-Rs), since they bind preferentially to N-methyl-D-aspartate (NMDA). NMDA is a chemical analog of aspartic acid; it normally does not occur in nature, and NMDA is not present in the brain. When molecules of NMDA contact neurons having NMDA-Rs, they strongly activate the NMDA-R (i.e., they act as a powerful receptor agonist), causing the same type of neuronal excitation that glutamate does. It has been known that excessive activation of NMDA-R plays a major role in a number of important central nervous system (CNS) disorders, while hypoactivity of NMDA-R has been implicated in several psychiatric diseases. In cultured hippocampal neurons, NMDA currents appear to be affected by PP1 and PP2A. However, it is not known whether that is due to an indirect effect mediated by PP1 or PP2A or a direct activity of the enzymes on the receptor.

NMDA-Rs contain an NR1 subunit and at least one of four different NR2 subunits (designated as NR2A, NR2B, NR2C, and NR2D), as well as NR3 subunits. NMDA-Rs are “ionotropic” receptors since they control ion channels. These ion channels allow ions to flow into a neuron, thereby activating the neuron, when the receptor is activated by glutamate, aspartate, or an agonist drug.

### SUMMARY OF THE INVENTION

The present invention provides methods for identifying a modulator of N-methyl-D-aspartate receptor (NMDA-R) signaling by detecting the ability of an agent to modulate the phosphatase activity of a protein serine/threonine phosphatase (PSTP) or to modulate the binding of the PSTP to NMDA-R. In some methods, the modulator is identified by detecting its ability to modulate the phosphatase activity of the PSTP. In some methods, the modulator is identified by detecting its ability to modulate the binding of the PSTP and the NMDA-R. In some methods, the PSTP is PP2A. In some methods, the NR2B subunit of NMDA-R is used to screen for the modulator.

Some of the methods of the present invention comprise the steps of (a) contacting (i) a test agent; (ii) PP2A; and (iii) a serine/threonine phosphorylated NMDA-R or a subunit thereof; wherein either or both of (ii) and (iii) is substantially pure or recombinantly expressed; (b) measuring the dephosphorylation activity of the PP2A on the NMDA-R or subunit; (c) comparing the dephosphorylation activity in the presence of the agent with the dephosphorylation activity in the absence of the agent, wherein a difference in the dephosphorylation activity identifies the agent as a modulator of NMDA-R signaling. In some of the methods, the NMDA-R and the PP2A exist in a PP2/NMDA-R-containing protein complex. In some methods, the test agent enhances the ability of the PP2A to dephosphorylate the NMDA-R. In some methods, the test agent inhibits the ability of the PP2A to dephosphorylate the NMDA-R. In still some of methods, the test agent modulates binding of the PP2A (or a functional derivative of PP2A) to NMDA-R (or a functional derivative of NMDA-R). In some of the methods, the test agent promotes or enhances the binding. In some of the methods, the test agent disrupts or inhibits the binding.

Some of the methods provided in the present invention comprise the steps of (a) obtaining a cell culture coexpressing the NMDA-R and PP2A; (b) introducing a nucleic acid molecule encoding a gene product into a portion of the cells; thereby producing cells comprising the nucleic acid molecule; (c) culturing the cells in (b) under conditions in which the gene product is expressed; and (d) measuring PP2A dephosphorylation activity on the NMDA-R in the cells in (c) and comparing the dephosphorylation activity with that of control cells into which the nucleic acid molecule has not been introduced, wherein a difference in dephosphorylation activity identifies the nucleic acid molecule as a modulator of NMDA-R signaling.

The invention further provides methods for treating diseases mediated by abnormal NMDA-R-signaling. Some of the methods comprise administering a modulator of a PP2A activity, thereby modulating the level of seine/threonine phosphorylation of the NMDA-R. In some methods, the modulator modulates the ability of PP2A to dephosphorylate NMDA-R. In some methods, the modulator modulates the ability of PP2A to bind to NMDA-R. In some methods, the modulator is a PP2A agonist, and the disease to be treated is selected from the group consisting of (i) ischemic stroke; (ii) head trauma or brain injury; (iii) Huntington's disease; (iv) spinocerebellar degeneration; (v) motor neuron diseases; (vi) epilepsy; (vii) neuropathic pain; (viii) chronic pain; and (ix) tolerance. In some other methods, the modulator is a PP2A antagonist, and the disease to be treated is selected from the group consisting of (i) schizophrenia; (ii) Alzheimer disease; (iii) dementia; (iv) psychosis; (v) depression; (vi) drug addiction; (vii) ethanol sensitivity, and (viii) attention disorders.

A further understanding of the nature and advantages of the present invention may be realized by reference to the remaining portions of the specification, the figures and claims.

All publications, GenBank deposited sequences, ATCC deposits, patents and patent applications cited herein are hereby expressly incorporated by reference in their entirety and for all purposes.

### BRIEF DESCRIPTION OF THE DRAWINGS

FIG. 1 shows co-immunoprecipitation of NMDA-R subunit NR2B with PP2A.

### DETAILED DESCRIPTION

The present invention is predicated in part on the discovery of a binding between the NR2B subunit of the NMDA-R and a serine/threonine protein phosphatase, PP2A. In accordance with the discovery, the present invention provides methods for identifying agonists and antagonists of a PSTP (e.g., PP2A) that modulates NMDA-R signaling, and for treating conditions mediated by abnormal NMDA-R signaling. The following sections provide guidance for making and using the compositions of the invention, and for carrying out the methods of the invention.

I. Definitions

Unless defined otherwise, all technical and scientific terms used herein have the same meaning as commonly understood by those of ordinary skill in the art to which this invention pertains. The following references provide one of skill with a general definition of many of the terms used in this invention: Singleton et al., DICTIONARY OF MICROBIOLOGY AND MOLECULAR BIOLOGY (2d ed. 1994); THE CAMBRIDGE DICTIONARY OF SCIENCE AND TECHNOLOGY (Walker ed., 1988); and Hale & Marham, THE HARPER COLLINS DICTIONARY OF BIOLOGY (1991). Although any methods and materials similar or equivalent to those described herein can be used in the practice or testing of the present invention, the preferred methods and materials are described. The following definitions are provided to assist the reader in the practice of the invention.

As used herein, the term “acute insult to the central nervous system” includes short-term events which pose a substantial threat of neuronal damage mediated by glutamate excitotoxicity. These include ischemic events (which involve inadequate blood flow, such as a stroke or cardiac arrest), hypoxic events (involving inadequate oxygen supply, such as drowning, suffocation, or carbon monoxide poisoning), trauma to the brain or spinal cord (in the form of mechanical or similar injury), certain types of food poisoning which involve an excitotoxic poison such as domoic acid, and seizure-mediated neuronal degeneration, which includes certain types of severe epileptic seizures. It can also include trauma that occurs to another part of the body, if that trauma leads to sufficient blood loss to jeopardize blood flow to the brain (for example, as might occur following a shooting, stabbing, or automobile accident).

The term “agent” includes any substance, molecule, element, compound, entity, or a combination thereof. It includes, but is not limited to, e.g., protein, polypeptide, small organic molecule, polysaccharide, polynucleotide, and the like. It can be a natural product, a synthetic compound, or a chemical compound, or a combination of two or more substances. Unless otherwise specified, the terms “agent”, “substance”, and “compound” can be used interchangeably.

As used herein, an “agonist” is a molecule which, when interacting with (e.g., binding to) a reference protein (e.g., PP2A, NMDA-R), increases or prolongs the amount or duration of the effect of the biological activity of the reference protein. By contrast, the term “antagonist,” as used herein, refers to a molecule which, when interacting with (e.g., binding to) a reference protein, decreases the amount or the duration of the effect of the biological activity of the reference protein (e.g., PP2A or NMDA-R). Agonists and antagonists may include proteins, nucleic acids, carbohydrates, antibodies, or any other molecules which decrease the effect of a reference protein. Unless otherwise specified, the term “agonist” can be used interchangeably with “activator”, and the term “antagonist” can be used interchangeably with “inhibitor”.

The term “analog” is used herein to refer to a molecule that structurally resembles a reference molecule but which has been modified in a targeted and controlled manner, by replacing a specific substituent of the reference molecule with an alternate substituent. Compared to the reference molecule, an analog would be expected, by one skilled in the art, to exhibit the same, similar, or improved utility. Synthesis and screening of analogs, to identify variants of known compounds having improved traits (such as higher potency at a specific receptor type, or higher selectivity at a targeted receptor type and lower activity levels at other receptor types) is an approach that is well known in pharmaceutical chemistry.

The term “biological preparation” refers to biological samples taken in vivo and in vitro (either with or without subsequent manipulation), as well as those prepared synthetically. Representative examples of biological preparations include cells, tissues, solutions and bodily fluids, a lysate of natural or recombinant cells.

As used herein, the term “functional derivative” of a native protein or a polypeptide is used to define biologically active amino acid sequence variants that possess the biological activities (either functional or structural) that are substantially similar to those of the reference protein or polypeptide. Thus, a functional derivative of PP2A must retain, among other activities, the ability to bind and dephosphorylate the NMDA-R. Similarly, a functional derivative of NMDA-R must be capable of binding to PP2A, and being phosphorylated by PP2A.

As used herein, the term “modulator of NMDA-R signaling” refers to an agent that is able to alter NMDA-R activity that is involved in the NMDA-R signaling pathways. The modulators include, but are not limited to, both “activators” and “inhibitors” of NMDA-R serine/threonine phosphorylation. An “activator” is a substance that enhances the serine/threonine phosphorylation level of NMDA-R, and thereby causes the NMDA receptor to become more active. The mode of action of the activator may be direct, e.g., through binding the receptor, or indirect, e.g., through binding another molecule which otherwise interacts with NMDA-R (e.g., PP2A). Conversely, an “inhibitor” decreases the serine/threonine phosphorylation of NMDA-R, and thereby causes NMDA receptor to become less active. The reduction may be complete or partial. As used herein, modulators of NMDA-R signaling would encompass PP2A antagonists and agonists.

The term “modulation” as used herein refers to both upregulation, (i.e., activation or stimulation), for example by agonizing; and downregulation (i.e. inhibition or suppression), for example by antagonizing, of a bioactivity (e.g., NMDA-R serine/threonine phosphorylation, PP2A serine/threonine phosphatase activity, PP2A binding to NMDA-R).

The term “NMDA-R hypofunction” is used herein to refer to abnormally low levels of signaling activity of NMDA-Rs on CNS neurons. For example, NMDA-R hypofunction may be caused by, e.g., abnormally low serine/threonine phosphorylation level of NMDA-R. NMDA-R hypofunction can occur as a drug-induced phenomenon. It can also occur as an endogenous disease process.

As used herein, the term “NMDA-R signaling” refers to its signal-transducing activities in the central nervous system that are involved in the various cellular processes such as neurodevelopment, neuroplasticity, and excitotoxicity. NMDA-R signaling affects a variety of processes including, but not limited to, neuron migration, neuron survival, synaptic maturation, learning and memory, and neurodegeneration.

As used herein, the term “PSTP modulator” includes both “activators” and “inhibitors” of a PSTP (e.g., PP2A) phosphatase activity on NMDA-R. In the case of PP2A, an “activator” is a substance which causes PP2A to become more active, and thereby decrease the serine/threonine phosphorylation level of NMDA-R. The mode of action of the activator may be direct, e.g., through binding PP2A, or indirect, e.g., through binding another molecule which otherwise interacts with PP2A. Conversely, an “inhibitor” of PP2A is a substance which causes PP2A to become less active, and thereby increase serine/threonine phosphorylation level of NMDA-R to a detectable degree. The reduction may be complete or partial, and due to a direct or an indirect effect.

As used herein, the term “polypeptide containing the catalytic subunit of PP2A” includes PP2A, and other polypeptides that contain the catalytic subunit of PP2A, or their derivatives, analogs, variants, or fusion proteins that can bind to NR2B. The term “polypeptide containing PP2A-binding site of NMDA-R” include NMDA-R that has at least an NMDA-R subunit (e.g., NR2B), and other polypeptides that contain the PP2A-binding site of NMDA-R or its subunit (e.g., the C-terminal fragment of NR2B as disclosed in the below Examples), or their derivatives, analogs, variants, or fusion proteins that can bind to PP2A.

As used herein, the term “PP2A/NMDA-R-containing protein complex” refers to protein complexes, formed in vitro or in vivo, that contain PP2A and NMDA-R. When only the binding between PP2A and NMDA-R is in concern, a polypeptide containing the catalytic subunit of PP2A and a polypeptide containing PP2A-binding site of NMDA-R can substitute for PP2A and NMDA-R respectively. However, when dephosphorylation of NMDA-R by PP2A is in concern, only a PP2A functional derivative and NMDA-R functional derivative can respectively substitute for PP2A and NMDA-R in the complex. In addition, the complex may also comprise other components, e.g., a protein serine/threonine kinase.

The terms “substantially pure” or “isolated,” when referring to proteins and polypeptides, e.g., a fragment of PP2A, denote those polypeptides that are separated from proteins or other contaminants with which they are naturally associated. A protein or polypeptide is considered substantially pure when that protein makes up greater than about 50% of the total protein content of the composition containing that protein, and typically, greater than about 60% of the total protein content. More typically, a substantially pure or isolated protein or polypeptide will make up at least 75%, more preferably, at least 90%, of the total protein. Preferably, the protein will make up greater than about 90%, and more preferably, greater than about 95% of the total protein in the composition.

A “variant” of a molecule such as PP2A or NMDA-R is meant to refer to a molecule substantially similar in structure and biological activity to either the entire molecule, or to a fragment thereof. Thus, provided that two molecules possess a similar activity, they are considered variants as that term is used herein even if the composition or secondary, tertiary, or quaternary structure of one of the molecules is not identical to that found in the other, or if the sequence of amino acid residues is not identical.

As used herein, “recombinant” has the usual meaning in the art, and refers to a polynucleotide synthesized or otherwise manipulated in vitro (e.g., “recombinant polynucleotide”), to methods of using recombinant polynucleotides to produce gene products in cells or other biological systems, or to a polypeptide (“recombinant protein”) encoded by a recombinant polynucleotide.

The term “operably linked” refers to functional linkage between a nucleic acid expression control sequence (such as a promoter, signal sequence, or array of transcription factor binding sites) and a second polynucleotide, wherein the expression control sequence affects transcription and/or translation of the second polynucleotide.

A “heterologous sequence” or a “heterologous nucleic acid,” as used herein, is one that originates from a source foreign to the particular host cell, or, if from the same source, is modified from its original form. Thus, a heterologous gene in a prokaryotic host cell includes a gene that, although being endogenous to the particular host cell, has been modified. Modification of the heterologous sequence can occur, e.g., by treating the DNA with a restriction enzyme to generate a DNA fragment that is capable of being operably linked to the promoter. Techniques such as site-directed mutagenesis are also useful for modifying a heterologous nucleic acid.

The term “recombinant” when used with reference to a cell indicates that the cell replicates a heterologous nucleic acid, or expresses a peptide or protein encoded by a heterologous nucleic acid. Recombinant cells can contain genes that are not found within the native (non-recombinant) form of the cell. Recombinant cells can also contain genes found in the native form of the cell wherein the genes are modified and re-introduced into the cell by artificial means. The term also encompasses cells that contain a nucleic acid endogenous to the cell that has been modified without removing the nucleic acid from the cell; such modifications include those obtained by gene replacement, site-specific mutation, and related techniques.

A “recombinant expression cassette” or simply an “expression cassette” is a nucleic acid construct, generated recombinantly or synthetically, that has control elements that are capable of affecting expression of a structural gene that is operably linked to the control elements in hosts compatible with such sequences. Expression cassettes include at least promoters and optionally, transcription termination signals. Typically, the recombinant expression cassette includes at least a nucleic acid to be transcribed (e.g., a nucleic acid encoding PP2A) and a promoter. Additional factors necessary or helpful in effecting expression can also be used as described herein. For example, transcription termination signals, enhancers, and other nucleic acid sequences that influence gene expression, can also be included in an expression cassette.

As used herein, “contacting” has its normal meaning and refers to combining two or more agents (e.g., two proteins, a polynucleotide and a cell, etc.). Contacting can occur in vitro (e.g., two or more agents [e.g., a test compound and a cell lysate] are combined in a test tube or other container) or in situ (e.g., two polypeptides can be contacted in a cell by coexpression in the cell, of recombinant polynucleotides encoding the two polypeptides), in a cell lysate”

Various biochemical and molecular biology methods referred to herein are well known in the art, and are described in, for example, Sambrook et al., Molecular Cloning: A Laboratory Manual, Cold Spring Harbor Press, N.Y. Second (1989) and Third (2000) Editions, and Current Protocols in Molecular Biology, (Ausubel, F. M. et al., eds.) John Wiley & Sons, Inc., New York (1987-1999).

II. Identification of Interaction of NMDA-R Subunits with PP2A by Yeast Two Hybrid Screening

The NMDA-R has been cloned and characterized (Hollmann et al., Ann. Rev. Neurosci. 17:31-108, 1994; McBain et al., Physiol. Rev. 74:723-760, 1994). The various serine/threonine phosphatases have also been described, e.g., in Herzig et al., Physiol. Rev. 80:173-210, 2000. The catalytic subunits of the various PSTP's share a high degree of sequence homology (see, e.g., Herzig et al., Physiol. Rev. 80:173-210, 2000). Many PSTP's or their catalytic subunits have been cloned and sequenced. These include PP1 (Bai et al., FASEB J. 2:3010-3016, 1988; Berndt et al., FEBS Lett. 223:340-346, 1987; Sasaki et al., Jpn J. Cancer Res. 81:1272-1280, 1990); PP2A-Cα (Arino et al., Proc. Natl. Acad. Sci. USA 85:4252-4256, 1988; Silva et al., FEBS Lett. 221:415-422, 1987); PP2A-Cβ (Arino et al., Proc. Natl. Acad. Sci. USA 85:4252-4256, 1988; and Silva et al., FEBS Lett. 226:176-178, 1987), PP2B (Giri et al., Biochem. Biophys. Res. Commun. 181:252-258, 1991; Kincaid et al., J. Biol. Chem. 265:11312-11319, 1990; Kuno et al., Biochem. Biophys. Res. Commun. 165:1352-1358, 1989; Muramatsu et al., Proc. Natl. Acad. Sci. USA 89:529-533, 1992); PP2C (Mann et al., Biochim. Biophys. Acta 1130:100-104, 1992; Tamura et al., Proc. Natl. Acad. Sci. USA 86:1796-1780, 1989; Terasawa et al., Arch. Biochem. Biophys. 307:342-349, 1993; Wenk et al., FEBS Lett. 297:135-138, 1992); PP3 (Honkanen et al., J. Biol. Chem. 266:6614-6619, 1991); PP4 (Brewis et al., EMBO J. 12:987-996, 1993); PP5 (Chen et al., EMBO J. 13:4278-4290, 1994); PP6 (Cohen et al., Trends Biochem. Sci. 22:245-251, 1997); and PP7 (Huang et al., J. Biol. Chem. 273:1462-1468, 1998).

Amino acid and nucleic acid sequences for PP2A, other serine/threonine protein phosphatases, and NMDA receptor subunits can readily be found in public databases (e.g., GenBank) and the scientific literature. For example, exemplary sequences for human clones have the following Genbank accession numbers: PP2A: AA195185 and AA019184; PP1: AA071347, AA071261, AA744617, and AA129790; NR2A: NM_{—}000833; NR2B: NM_{—}000834; NR2C: NM_{—}000835; NR2D: NT_{—}011190; NR1: NM_{—}000832. Exemplary sequences for rat clones have the following Genbank accession numbers: NR2A: M91561; NR2B: M91562; NR2C: M91563; NR2D: D13213; NR1: X63255. Additional sequence information is readily available. Further, polynucleotides encoding proteins of interest can be obtained using sequence information by routine methods (e.g., cloning or amplification using probes or primers designed from the sequences). These clones, their homologs and derivatives can be used in the present invention.

As detailed in the Examples, infra, the present inventors have identified an interaction between PP2A and NR2B using a yeast two-hybrid screening system. This interaction was further confirmed by co-immunoprecipitation of PP2A and NMDA-R subunit (see the Examples). These results strongly indicate that PP2A is involved in the dephosphorylation of NMDA-R, and that the PP2A/NR2B interaction plays a role in modulation of PP2A phosphatase activity on NMDA-R. For example, PP2A can bind to NR2B and dephosphorylate NR1. In accordance with the present invention, the physiological significance of the PP2A/NR2B interaction can be examined by various methods including phosphorylation experiments, electrophysiology, and co-localization approaches. Subsequent to the above-noted findings of the present inventors, it was reported that NMDA-R subunit NR3A also physically interacts with PP2A, and that the interaction leads to increased dephosphorylation of NR1 subunit (Chan and Sucher, J. Neurosci. 21:7985-92, 2001).

III. Screening for Modulators of NMDA-R Signaling

The present invention provides methods for identifying modulators of NMDA-R signaling. The NMDA-R modulators are identified by detecting the ability of an agent to modulate an activity of a serine/threonine protein phosphatase (PSTP) that is capable of dephosphorylating NMDA-R. The modulated activities of the PSTP include, but are not limited to, its phosphatase activity or its binding to NMDA-R.

Preferably, the PSTP used for screening NMDA-R modulators is PP2A, its catalytic subunit, or a fragment thereof. For example, PP2A used in the screening can be encoded by a polynucleotide having the sequence of SEQ ID NO: 1 or SEQ ID NO:2. Alternatively, other PP2A polynucleotide sequences as shown in Table 1 below can also be used to obtain PP2A polypeptides for screening NMDA-R modulators.

In some methods, the NMDA-R modulators are screened for their ability to modulate PP2A phosphatase activity. In some methods, the NMDA-R modulators are identified by detecting their ability to promote or suppress the binding of PP2A and NMDA-R.

A. Identification of NMDA-R Modulators by Monitoring Dephosphorylation or Other Activities of NMDA-R

1. Modulation of NMDA-R Dephosphorylation

In some methods, NMDA-R modulators of the present invention are identified by monitoring their ability to modulate the phosphatase activity of a PSTP (e.g., PP2A). The modulators of NMDA-R can be identified by monitoring the effects of a test agent on the dephosphorylation of a substrate by a PSTP. For example, inhibition of PP1, PP2A or PP3 can be assessed by methods known to one of ordinary skill in the art. Suitable assays are described, for example by Honkanen et al. (1994) Toxicon 32:339 and Honkanen et al. (1990) J. Biol. Chem. 265: 19401. Other assays have been described, e.g., in U.S. Pat. Nos. 5,914,242, and 6,066,485. In these methods, phosphatase activity is determined by quantifying the ³²p released from a ³²P-labeled substrate such as phosphohistone or phosphorylase-α. Other suitable substrates include ³²P-labeled bovine brain myelin basic protein (MBP) (see, e.g., U.S. Pat. No. 5,916,749) and radiolabeled phosphorylated peptide substrate. The latter is derived from the serine phosphorylation site sequence of the RII subunit of cAMP-dependent protein kinase (Aldape et al., J. Biol. Chem. 267:16029-16032, 1992; and U.S. Pat. No. 5,978,740.

In other suitable assays, the ability of a test agent to inhibit the activity of a PSTP (e.g., PP2A) can be assessed in a 96-well microtiter plates using the substrate fluorescein diphosphate as described, e.g., in U.S. Pat. No. 6,040,323. In these assays, fluorescence emission from the substrate is measured spectrofluorometrically, e.g., with Perceptive Biosystems Cytofluor II (Framingham, Mass.). The rate of increase in fluorescence due to formation of dephosphorylated substrate is proportional to phosphatase activity. Other assays that can be used to measure the modulatory effects of a test agent on PSTP's include, e.g., calorimetric assays as described, e.g., U.S. Pat. No. 5,441,880.

Regardless of the assay used, the effect of a test agent is determined by comparing the phosphatase activity of a PSTP (e.g., PP2A) in the presence of the test agent with a control (i.e., phosphatase activity in the absence of the test agent). A change in the phosphatase activity of a PSTP (e.g., PP2A) when a test agent is present relative to the control provides a measure of the ability of the test agent to modulate the PSTP phosphatase activity. A PSTP (e.g., PP2A) antagonist is identified if the presence of the test agent results in a decreased phosphatase activity. Conversely, an increased phosphatase activity indicates that the test agent is a PSTP agonist.

The PSTP (e.g., PP2A) used in the assays can be obtained from various sources. In some methods, PP2A used in the assays is purified from cellular or tissue sources, e.g., by immunoprecipitation with specific antibodies. In some methods, as described below, PP2A is purified by affinity chromatography utilizing specific interactions of PP2A with known protein motifs, e.g., the interaction of the catalytic subunit of PP2A with NR2B. In some methods, PP2A, either holoenzyme or enzymatically active parts of it, is produced recombinantly either in bacteria or in eukaryotic expression systems. The recombinantly produced variants of PP2A can contain short protein tags, such as immunotags (HA-tag, c-myc tag, FLAG-tag) or 6×His-tag, which could be used to facilitate the purification of recombinantly produced PP2A using immunoaffinity or metal-chelation-chromatography, respectively. In addition to the substrates described in the art, NMDA-R, a functional derivative of NMDA-R, or the NR2B subunit can also be used to prepare phosphorylated substrates for a PSTP (e.g., PP2A). In some methods, the substrates can be purified from a tissue (such as immunoprecipitated NR2B from rat brain). In other embodiments, the substrates are recombinantly expressed proteins. Examples of recombinant substrates include, but are not limited to, NR2B fusion proteins expressed in E. coli, yeast, insect cells, or mammalian expression systems.

Methods and conditions for expression of recombinant proteins are well known in the art. See, e.g., Sambrook, supra, and Ausubel, supra. Typically, polynucleotides encoding the phosphatase and/or substrate used in the invention are expressed using expression vectors. Expression vectors typically include transcriptional and/or translational control signals (e.g., the promoter, ribosome-binding site, and ATG initiation codon). In addition, the efficiency of expression can be enhanced by the inclusion of enhancers appropriate to the cell system in use. For example, the SV40 enhancer or CMV enhancer can be used to increase expression in mammalian host cells. Typically, DNA encoding a polypeptide of the invention is inserted into DNA constructs capable of introduction into and expression in an in vitro host cell, such as a bacterial (e.g., E. coli, Bacillus subtilus), yeast (e.g., Saccharomyces), insect (e.g., Spodoptera frugiperda), or mammalian cell culture systems. Mammalian cell systems are preferred for may applications. Examples of mammalian cell culture systems useful for expression and production of the polypeptides of the present invention include human embryonic kidney line (293; Graham et al., 1977, J. Gen. Virol. 36:59); CHO (ATCC CCL 61 and CRL 9618); human cervical carcinoma cells (HeLa, ATCC CCL 2); and others known in the art. The use of mammalian tissue cell culture to express polypeptides is discussed generally in Winnacker, FROM GENES TO CLONES (VCH Publishers, N.Y., N.Y., 1987) and Ausubel, supra. In some embodiments, promoters from mammalian genes or from mammalian viruses are used, e.g., for expression in mammalian cell lines. Suitable promoters can be constitutive, cell type-specific, stage-specific, and/or modulatable or regulatable (e.g., by hormones such as glucocorticoids). Useful promoters include, but are not limited to, the metallothionein promoter, the constitutive adenovirus major late promoter, the dexamethasone-inducible MMTV promoter, the SV40 promoter, and promoter-enhancer combinations known in the art.

Phosphorylated form of the substrate proteins or polypeptides can be phosphorylated using serine/threonine kinases well known in the art, e.g., the human protein kinase as described in U.S. Pat. No. 6,096,308 or human growth factor receptor binding protein (HSTPK) as described in U.S. Pat. No. 6,162,431. Additional serine/threonine protein kinases have been described in Edelman et al., Protein serine/threonine kinases, Annu Rev Biochem 56:567-613,1987; Shi et al., The serine, threonine, and/or tyrosine-specific protein kinases and protein phosphatases of prokaryotic organisms: a family portrait, FEMS Microbiol Rev, 22:229-53, 1998; Hanks et al., The Protein Kinase Family: Conserved Features and Deduced Phylogeny of the Catalytic Domains, Science 241:42 (1988); Cohen, Dissection of Protein Kinase Cascades That Mediate Cellular Response to Cytokines and Cellular Stress, Advances in Pharmacology, 36:15 (1996); and U.S. Pat. Nos. 6,171,841, 6,133,006, 6,093,728, 6,034,228, 6,013,500, 5,965,365, 5,962,265, 5,958,748, and 5,650,501. Using these kinases, various methods routinely practiced in the art can be carried out to phosphorylate the substrates, e.g., as described, e.g., in Honkanen et al. (1990) J. Biol. Chem. 265: 19401; U.S. Pat. Nos. 5,856,161, 6,162,431, and 6,096,308.

2. Modulating NMDA-R Dephosphorylation by Monitoring its Channel Activity

NMDA-Rs are ligand-gated cation channels. Modulation of NMDA-R dephosphorylation can also be examined by monitoring NMDA-R channel activity.

In some methods, NMDA-R channel activity (e.g., glutamate—induced calcium influx) is monitored by measuring NMDA currents in the presence of a test agent in cultured hippocampal or cerebellar granule neurons using patch-clamp techniques and confocal scanning microscopy (see, e.g., Wang et al., Nature 369:230-232, 1994; and Medina et al. J. Physiol. 495:411-27, 1996). The patch-clamp technique can also be used to study whole-cell current through heteromeric NR1—NR2A and NR1-NR2B subunit combinations of NMDA channels transiently expressed in human embryonic kidney cells (see, e.g., Medina et al., J. Physiol. 482:567-73, 1995).

In some methods, NMDA-R channel activity is monitored by analyzing receptor desensitization in outside-out patches from cultured neurons as described, e.g., in Tong et al. (J. Neurophysiol. 72:754-61, 1994). In still some methods, modulation of NMDA-R dephosphorylation is examined by monitoring duration of single NMDA channel openings, bursts, clusters and superclusters. This can be performed in cell-attached recordings in acutely dissociated adult rat dentate gyrus granule cells, as described, e.g., in Lieberman et al. (Nature 369:235-9, 1994).

In some methods, NMDA-R channel activity can be monitored by measuring increased calcium influx upon activation of glutamate. Thus PP2A inhibitors may lead to increased Ca2+ entry and NMDA-R activity. Measurements can be done in presence/absence of compounds in primary hippocampal neurons as well as in transiently transfected HEK293 cells expressing NR1 and NR2 subunits, e.g., using FLEX station/Flipper or Ca2+ Imaging. The Molecular Devices FLEX station is a scanning fluorometer coupled with a fluid transfer system that allows the measurement of rapid, real time fluorescence changes in response to application of compunds. As the function of NMDA receptors depends critically upon their ability to act as calcium channels upon activation, the FLEX station in combination with calcium indicator dyes can be used to measure NMDA receptor activity. This allows investigation of roles of interacting proteins in the modulation of both the magnitude and kinetics of NMDA receptor mediated calcium influx and screening for compounds that are able to modulate the functional properties of NMDA receptors.

It is also contemplated by the present invention to infect primary neurons with either Adenovirus, Sindbisvirus or others vectors expressing PP2A and inactive mutations thereof. NMDA-R function can be monitored accordingly. Receptor localization can also be examined, e.g., by immunocytochemistry.

B. Screening for NMDA-R Modulators by Monitoring Binding of PSTP and NMDA-R

Modulation of binding of a PSTP (e.g., PP2A) and NMDA-R can also affect dephosphorylation of NMDA-R by the PSTP. Therefore, agents identified from monitoring phosphorylation level of NMDA-R using the assays described above can also encompass agents that modulate NMDA-R phosphorylation by affecting the binding of a PSTP (e.g., PP2A) and NMDA-R. In some methods of the present invention, NMDA-R modulators are identified by directly screening for agents that promote or suppress the binding of a PSTP (e.g., PP2A) and NMDA-R. Agents thus identified may be further examined for their ability to modulate NMDA-R serine/threonine phosphorylation, using methods described above or standard assays well know in the art.

1. Assays Based on Two-Hybrid Screening System

A variety of binding assays are useful for identifying agents that modify the interaction between a PSTP (e.g., PP2A or its catalytic subunit) and NMDA-R or NR2B. In some methods, two-hybrid based assays are used.

i) Yeast Two-Hybrid Assay

The cDNAs encoding the C-terminal portion, typically at least 100, 200, 400, or 600 C-terminal amino acid residues, of NR2B, and at least the catalytic subunit of a PSTP (e.g., PP2A) can be cloned into yeast two-hybrid vectors encoding the DNA binding domain and DNA activation domain, respectively, or vice-versa. The yeast two-hybrid used is based on the yeast GAL4 transcriptional system (Song & Fields, Nature 340: 245-246, 1989), the Sos-Ras complementation system (Aronheim et al., Mol. Cell. Biol. 17: 3094-3102, 1997), the bacterial LexA transcriptional system (Current Protocols in Mol. Biol., Ausubel et al. Eds, 1996, New York), or any other system of at least equal performance. Reporter gene constructs, such as α- or β-galactosidase, β-lactamase, or green fluorescent protein (GFP, see, Tombolini et al., Methods Mol. Biol. 102: 285-98, 1998; Kain et al., Methods Mol. Biol. 63: 305-24, 1997), are produced using necessary regulatory elements from promoter regions of above-mentioned transcription factors. Alternatively, modular signaling molecules are engineered to be brought together by the interaction between NR2B and PP2A in the Sos-Ras complementation-based yeast two-hybrid system. These constructs are transiently or stably transformed into a yeast strain to be used in the screen.

In some methods, the GAL4 system is used to screen agents that modulate the binding of a PSTP (e.g., PP2A) and NMDA-R. DNA binding domain vector containing the C-terminal portion of NR2B and DNA activation domain vector containing the catalytic subunit of PP2A are cotransformed into the same yeast strain which carries one of the reporters. The interaction between a PSTP (e.g., PP2A) and NMDA-R activates the expression of the reporter gene. The yeast culture in which the reporter genes are expressed is divided in equal amounts to 96- or 384-well assay plates. The levels of α- or β-galactosidase, β-lactamase are measured by quantifying their enzymatic activity using colorimetric substrates, such as orthomethylphenylthiogalactoside (OMTP) or X-gal; the levels of GFP are assessed fluorometrically. Pools of agents or individual agents are added to yeast cultures in wells and the levels of inhibition or facilitation of the interaction by the agents are determined from the levels of the reporter gene activity. Agents which decrease the reporter gene expression are antagonists of the interaction between a PSTP (e.g., PP2A) and NR2B. In contrast, agents which facilitate the reporter gene expression are agonists of the interaction between a PSTP (e.g., PP2A) and NR2B.

2. Other Binding Assays

In some methods of the invention, agents (e.g., peptides) that bind to the catalytic subunit of a PSTP (e.g., PP2A) with high affinity are identified by phage display, oriented peptide library approach (Songyang et al., Science 275: 73-77, 1997) or lacI repressor system (Stricker et al., Methods in Enzymology 303: 451-468, 1999). These peptides are further screened for their ability to modulate the interaction between a PSTP (e.g., PP2A) and NR2B.

In some methods, modulators of the interaction between a PSTP (e.g., PP2A) and NR2B are identified by detecting their abilities to either inhibit the PSTP and NMDA-R from binding (physically contacting) each other or disrupts a binding of a PSTP (e.g., PP2A) and NMDA-R that has already been formed. The inhibition or disruption can be either complete or partial. In some methods, the modulators are screened for their activities to either promote the PSTP and NMDA-R to bind to each other, or enhance the stability of a binding interaction between the PSTP and NMDA-R that has already been formed. In either case, some of the assays discussed above for identifying agents which modulate the NMDA-R phosphorylation level may be directly applied or readily modified to monitor the effect of an agent on the binding of NMDA-R and the PSTP. For example, a cell transfected to coexpress PP2A and NMDA-R or NR2B, in which the two proteins interact to form NMDA-R/PP2A-containing complex, is incubated with an agent suspected of being able to inhibit this interaction, and the effect on the interaction measured. In some methods, a polypeptide containing the catalytic subunit of PP2A and a polypeptide containing PP2A-binding site of NMDA-R can substitute for the intact PP2A and NMDA-R proteins, respectively, in the NMDA-R/PP2A-containing protein complexes. Any of a number of means, such as co-immunoprecipitation, can be used to measure the interaction and its disruption.

C. Screening for NMDA-R Modulators Using PP2A and NMDA-R Functional Derivatives/Subunits or Other PSTP's

Although the foregoing assays or methods are described with reference to PP2A and NMDA-R, the ordinarily skilled artisan will appreciate that functional derivatives or subunits of PP2A and NMDA-R can also be used. For example, in various methods, NR2B can be used to substitute for an intact NMDA-R in assays for screening agents that modulate binding of PP2A and NMDA-R. In some methods, an NMDA-R functional derivative can be used for screening agents which modulate PP2A phosphatase activity on NMDA-R. In some methods, rather than the NR2B subunit, the NR2A, NR2C, or NR2D subunit can be used. In still some methods, a polypeptide containing the catalytic subunit of PP2A can be used for screening agents which modulate the binding of PP2A and NMDA-R.

In addition, while PP2A is the preferred serine/threonine protein phosphatase for practicing the presently claimed methods, other PSTP's can also be used to screen NMDA-R modulators. For example, PP1 has been implicated to be involved in regulation of NMDA-R receptor activity (Wang et al., Nature 369:230-232, 1994; and Westphal. et al, Science, 285:93-6, 1999). Thus, PP1 can be used to substitute for PP2A in the various methods described above.

Further, in various methods, functional derivatives of the PSTP's (e.g., PP2A or PP1) that have amino acid deletions and/or insertions and/or substitutions (e.g., conservative substitutions) while maintaining their catalytic activity and/or binding capacity are used for the screening of agents. Similarly, NMDA-R mutants that maintain serine/threonine phosphorylation activity and PP2A-binding activity can be used. A functional derivative is prepared from a naturally occurring or recombinantly expressed PP2A and NMDA-R by proteolytic cleavage followed by conventional purification procedures known to those skilled in the art. Alternatively, the functional derivative is produced by recombinant DNA technology by expressing only fragments of PP2A or NMDA-R in suitable cells. In some methods, the partial receptor or phosphatase polypeptides are expressed as fusion polypeptides. It is well within the skill of the ordinary practitioner to prepare mutants of naturally occurring NMDA-R or PP2A proteins that retain the desired properties, and to screen the mutants for binding and/or enzymatic activity. NR2B derivatives that can be dephosphorylated typically comprise the cytoplasmic domain of the polypeptides, e.g., the C-terminal 597 amino acids or a fragment thereof. However, the present inventors have also found that the last 105 amino acid residues of the NR2B C-terminus do not interact with PP2A, based on yeast specificity tests (data not shown). Thus, a NR2B fragment lacking this portion can be used to analyze PP2A and NR2B interaction. PP2A deletion constructs carrying only the catalytic subunit are able to bind the C-terminal 600 amino acids of NR2B in vitro. Functional derivatives of PP2A that bind the NMDA-R or that retain enzymatic (dephosphorylation) activity will usually include the catalytic subunit.

In some methods, cells expressing PP2A and NMDA-R can be used as a source of PP2A and/or NMDA-R, crude or purified, for testing in these assays. The cells can be genetically engineered to coexpress PP2A and NMDA-R. The cells can also be used as host cells for the expression of other recombinant molecules with the purpose of bringing these molecules into contact with PP2A and/or NMDA-R within the cell.

IV. Therapeutic Applications and Pharmaceutical Compositions

It is well known in the art that NMDA-R agonists and antagonists can be used to treat symptoms caused by abnormal NMDA-R signaling (e.g., acute insult of the central nervous system (CNS)). Methods of treatment using pharmaceutical composition comprising NMDA agonists and/or NMDA antagonists have been described, e.g., in U.S. Pat. No. 5,902,815. As discussed in detail below, the present invention provides pharmaceutical compositions containing PSTP (e.g., PP2A) antagonists and/or agonists that modulate NMDA-R serine/threonine phosphorylation. Such agonists and antagonists include, but are not limited to, agents that interfere with PP2A gene expression, agents that modulate the ability of PP2A to bind to NMDA-R or to dephosphorylate NMDA-R. In some methods, a PP2A antisense oligonucleotide is used as a PP2A antagonist in the pharmaceutical compositions of the present invention.

In addition to NMDA-R agonists or antagonists that can be identified in accordance with the present invention, PSTP agonists or antagonists known in the art can also be used in the methods of the present invention for treating NMDA-R related disorders. For example, inhibitors that inhibit PSTP (e.g., PP2A) phosphatase activity can be useful as NMDA-R signaling modulators. Potent inhibitors of the serine/threonine phosphatases have been identified, including proteins designated Inhibitor-1, Inhibitor-2, DARPP-32, and NIPP-1 (see, e.g., Li et al., Biochemistry 34:1988-96, 1995; Li et al., Biochemistry 35:6998-7002, 1996; U.S. Pat. No. 6,040,323; and Honkanen et al. in Protein Kinase C, Kuo, ed., Oxford Univ. Press, Oxford, 1994). Non-protein inhibitors have also been identified as potent inhibitors of the serine/threonine phosphatases (see, e.g., Fujiki et al. (1993) Gazz. Chim. Ital. 123: 309. For example, okadaic acid, a polyether fatty acid produced by several species of marine dinoflagellates, reversibly inhibits the catalytic subunits of serine/threonine phosphatase subtypes PP1, PP2A and PP3. Calyculin A, a cytotoxic component of the marine sponge Discodermia calyx, also has an inhibitory activity on PP1, PP2A and PP3.

In addition to PSTP antagonists, activators of PSTP's are also known in the art. For example, 2,3-Butanedione monoxime (BMD) (Zimmermann et al., Naunyn-Schmiedeberg's Arch. Pharmacol. 354:431-436, 1996) and Sphingosine derivatives such as ceramide (J. Biol. Chem. 268:15523-15530, 1993) have been shown to be able to activate PSTP's. Polylysine, protamine, polybrene, and histone H1 are also known to activate PP2A (Erdodi et al., Biochim. Biophys. Acta. 827:23-29, 1985; and Pelech et al., Eur. J. Biochem. 148:245-251, 1985).

A. Therapeutic Application of the Present Invention

Abnormal NMDA-R activity elicited by endogenous glutamate is implicated in a number of important CNS disorders. In one aspect, the present invention provides modulators of a PSTP (e.g., PP2A) that, by modulating serine/threonine phosphorylation level of NMDA-R, can treat or alleviate symptoms mediated by abnormal NMDA-R signaling.

One important use for NMDA antagonist drugs involves the ability to prevent or reduce excitotoxic damage to neurons. In some methods, the PSTP agonists of the present invention, which promote the dephosphorylation of NMDA-R, are used to alleviate the toxic effects of excessive NMDA-R signaling. In certain other methods, PSTP antagonists of the present invention, which function as NMDA-R agonists, are used therapeutically to treat conditions caused by NMDA-R hypo-function, i.e., abnormally low levels of NMDA-R signaling in CNS neurons. NMDA-R hypofunction can occur as an endogenous disease process. It can also occur as a drug-induced phenomenon, following administration of an NMDA antagonist drug.

B. Specific Examples of Diseases and Disorders to be Treated

Excessive glutamatergic signaling has been causatively linked to the excitotoxic cell death during an acute insult to the central nervous system such as ischemic stroke (Choi et al., Annu Rev Neurosci. 13: 171-182, 1990; Muir & Lees, Stroke 26: 503-513, 1995). Excessive glutamatergic signaling via NMDA receptors has been implicated in the profound consequences and impaired recovery after the head trauma or brain injury (Tecoma et al., Neuron 2:1541-1545, 1989; McIntosh et al., J. Neurochem. 55:1170-1179, 1990). NMDA receptor-mediated glutamatergic hyperactivity has also been linked to the process of slow degeneration of neurons in Parkinson's disease (Loopuijt & Schmidt, Amino Acids, 14: 17-23, 1998) and Huntington's disease (Chen et al., J. Neurochem. 72:1890-1898, 1999). Further, elevated NMDA-R signaling in different forms of epilepsy have been reported (Reid & Stewart, Seizure 6: 351-359, 1997).

Accordingly, PSTP agonists of the present invention are used for the treatment of these diseases or disorders by stimulating the NMDA receptor-associated serine/threonine phosphatase activity (such as that of PP2A) or by promoting the binding of the PSTP (e.g., PP2A) to the NMDA receptor complex.

The PSTP agonists (NMDA-R antagonists) of the present invention can also be used to treat diseases where a mechanism of slow excitotoxicity has been implicated (Bittigau & Ikonomidou, J. Child. Neurol. 12: 471-485, 1997). These diseases include, but are not limited to, spinocerebellar degeneration (e.g., spinocerebellar ataxia), motor neuron diseases (e.g., amyotrophic lateral sclerosis (ALS)), mitochondrial encephalomyopathies, and depression. The PSTP agonists of the present invention can also be used to alleviate neuropathic pain, or to treat chronic pain without causing tolerance or addiction (see, e.g., Davar et al., Brain Res. 553: 327-330, 1991).

On the other hand, NMDA-R hypofunction have been causatively linked to schizophrenic symptoms (Tamminga, Crit. Rev. Neurobiol. 12: 21-36, 1998; Carlsson et al., Br. J. Psychiatry Suppl.: 2-6, 1999; Corbett et al., Psychopharmacology (Berl). 120: 67-74, 1995; Mohn et al., Cell 98: 427-436, 1999) and various forms of cognitive deficiency or mental disorders, such as dementias (e.g., senile and HIV-dementia), depression (Hrabetova et al., J Neurosci 20: RC81, 2000; Menniti et al., Neuropharmacology, 39: 1147-55, 2000; and Maruoka et al., Gen Pharmacol. 29: 645-9, 1997), and Alzheimer's disease (Lipton, Annu. Rev. Pharmacol. Toxicol. 38:159-177, 1998; Ingram et al., Ann. N.Y. Acad. Sci. 786: 348-361, 1996; Müller et al., Pharmacopsychiatry. 28: 113-124, 1995). In addition, NMDA-R hypofunction is also linked to psychosis and drug addiction (Javitt & Zukin, Am J Psychiatry. 148: 1301-8, 1991). Further, NMDA-R hypofunction is also associated with ethanol sensitivity (Wirkner et al., Neurochem. Int. 35: 153-162, 1999; Yagi, Biochem. Pharmacol. 57: 845-850, 1999).

Using PSTP antagonist (NMDA-R agonists) described herein, the present invention provides methods for the treatment of Schizophrenia, psychosis, cognitive deficiencies, drug addiction, and ethanol sensitivity by antagonizing the activity of the NMDA-R-associated PSTP's, and that of PP2A in particular, or by inhibiting the interaction between a PSTP (e.g., PP2A) and the NR2B subunit.

C. Dosages and Modes of Administration

The PSTP agonists and antagonists of the present invention can be directly administered under sterile conditions to the host to be treated. However, while it is possible for the active ingredient to be administered alone, it is often preferable to present it as a pharmaceutical formulation. Formulations typically comprise at least one active ingredient together with one or more acceptable carriers thereof. Each carrier should be both pharmaceutically and physiologically acceptable in the sense of being compatible with the other ingredients and not injurious to the patient. For example, the bioactive agent is complexed with carrier proteins such as ovalbumin or serum albumin prior to their administration in order to enhance stability or pharmacological properties such as half-life. Furthermore, therapeutic formulations of this invention are combined with or used in association with other therapeutic agents.

The therapeutic formulations are delivered by any effective means which could be used for treatment. Depending on the specific NMDA-R antagonist and/or NMDA-R agonist being used, the suitable means include but are not limited to oral, rectal, nasal, pulmonary administration, or parenteral (including subcutaneous, intramuscular, intravenous and intradermal) infusion into the bloodstream.

Therapeutic formulations are prepared by any methods well known in the art of pharmacy. See, e.g., Gilman et al (eds.) (1990) Goodman and Gilman's: The Pharmacological Bases of Therapeutics (8th ed.) Pergamon Press; and (1990) Remington's Pharmaceutical Sciences (17th ed.) Mack Publishing Co., Easton, Pa.; Avis et al (eds.) (1993) Pharmaceutical Dosage Forms: Parenteral Medications Dekker, N.Y.; Lieberman et al. (eds.) (1990) Pharmaceutical Dosage Forms: Tablets Dekker, N.Y.; and Lieberman et al (eds.) (1990) Pharmaceutical Dosage Forms: Disperse Systems Dekker, N.Y. The therapeutic formulations can conveniently be presented in unit dosage form and administered in a suitable therapeutic dose. The preferred dosage and mode of administration of a PP2A agonist and/or antagonist will vary for different patients, depending upon factors that will need to be individually reviewed by the treating physician. As a general rule, the quantity of a PP2A agonist and/or antagonist administered is the smallest dosage which effectively and reliably prevents or minimizes the conditions of the patients.

A suitable therapeutic dose is determined by any of the well known methods such as clinical studies on mammalian species to determine maximum tolerable dose and on normal human subjects to determine safe dosage. In human patients, since direct examination of brain tissue is not feasible, the appearance of hallucinations or other psychotomimetic symptoms, such as severe disorientation or incoherence, should be regarded as signals indicating that potentially neurotoxic damage is being generated in the CNS by NMDA-R antagonist. Additionally, various types of imaging techniques (such as positron emission tomography and magnetic resonance spectroscopy, which use labeled substrates to identify areas of maximal activity in the brain) may also be useful for determining preferred dosages of NMDA-R agonists for use as described herein, with or without NMDA-R antagonists.

It is also desirable to test rodents or primates for cellular manifestations in the brain, such as vacuole formation, mitochondrial damage, heat shock protein expression, or other pathomorphological changes in neurons of the cingulate and retrosplenial cerebral cortices. These cellular changes can also be correlated with abnormal behavior in laboratory animals.

Except under certain circumstances when higher dosages may be required, the preferred dosage of a PP2A agonist and/or antagonist will usually lie within the range of from about 0.001 to about 1000 mg, more usually from about 0.01 to about 500 mg per day. It should be understood that the amount of any such agent actually administered will be determined by a physician, in the light of the relevant circumstances that apply to an individual patient (including the condition or conditions to be treated, the choice of composition to be administered, including the particular PSTP agonist or the particular PSTP antagonist, the age, weight, and response of the individual patient, the severity of the patient's symptoms, and the chosen route of administration). Therefore, the above dosage ranges are intended to provide general guidance and support for the teachings herein, but are not intended to limit the scope of the invention.

V. Methods for Purification of PSTP

The present invention provides methods for purification of the PP2A protein or a polypeptide containing the catalytic subunit of PP2A. Specifically, identification of the binding between PP2A and NR2B allows affinity purification of PP2A or polypeptide containing the catalytic subunit of PP2A, using methods well known in the art. For standard methods for affinity purification of proteins, see, e.g., Protein purification, principles, high resolution methods and applications, Janson and Ryden eds., 1989; Scopes, R. K., Chapter 3, Protein Purification, Principles and Practice, 2nd Ed., Springer-Verlag, New York, 1987; Deutscher, M. P., Guide to Protein Purification, Academic Press, 1990, pp. 174-193.

In some methods, a polypeptide containing the PP2A-binding site of NMDA-R is attached to a solid matrix (e.g., CNBr-activated Sepharose). The remaining active sites on the matrix are blocked with a suitable agent (e.g., BSA). After applying the biological preparation to the matrix and allowing binding of PP2A to the polypeptide containing the PP2A-binding site of NMDA-R on the matrix, the matrix is washed to remove non-specific binding molecules from the matrix. PP2A or polypeptide containing the catalytic subunit of PP2A can then be eluted from the matrix and recovered according to methods well known in the art.

### VI. EXAMPLES

The following examples are provided to further illustrate the present invention. They are not included to limit the invention in any way. Many modifications and variations of this invention can be made without departing from its spirit and scope, as will be apparent to those skilled in the art.

### Example 1

### Identification of the NR2/PP2A Binding Using Yeast Two-Hybrid Screen

A yeast two-hybrid screen was carried out as follows. A commercially available adult rat brain cDNA library in the pACT2 vector pretransformed to the Y187 yeast strain (Clontech) was used. The cDNA corresponding to the 597 C-terminal amino acid residues of the NR2B subunit was fused with GAL4 BD by cloning it into the pGBKT7 vector (Clontech). The resulting GAL4BD-NR2B plasmid (bait) was transformed to AH109 strain (Clontech) to screen for the NR2B C-terminus interacting proteins in the rat brain cDNA library. The Y 187 cells were mated in rich (YPD) medium for 20 hours with at least a ten-fold excess of AH109 cells carrying the bait vector. For selection of interactors, the yeast cells were plated for selection after mating on the solid yeast medium depleted of histidine, adenine, tryptophan, and leucine, and in the presence of X-gal. The AD plasmids from only those colonies which survived the double growth-selection and yielded strong colorimetric reaction in the β-galactosidase assay were further analyzed by DNA sequencing.

One yeast colony thus identified, designated YH04A_CO₂, contained a cDNA clone which has a high degree of sequence identity to PP2A. As shown in Table 1, the 347 bp DNA sequence of YH04A_CO₂ (SEQ ID NO: 1) has a sequence identity around 97-99% to various known PP2A catalytic subunit coding sequences.

TABLE 1

Alignment of YH04A_C02 Sequence (“Query”; SEQ ID NO: 1) with Known PP2A
Sequences (“Subject”; SEQ ID NOS: 8-12)

| | |
|---|---|
| 1. | With Rat mRNA for protein phosphatase-2A catalytic subunit |

(Accession No. X14159.1; SEQ ID NO: 8)
Identities = 306/307 (99%), Positives = 306/307 (99%)

| | | | |
|---|---|---|---|
| Query: | 41 | CGACAGCAGACAGATCACACAAGTTTATGGTTTCTACGATGAGTGTTTAAGGAAATACGG | 100 |
| Sbjct: | 467 | CGAGAGCAGACAGATCACACAAGTTTATGGTTTCTACGATGAGTGTTTAAGGAAATACGG | 526 |
| Query: | 101 | AAATGCAAATGTTTGGAAATACTTCACAGACCTTTTTGACTACCTTCCTCTCACTGCCTT | 160 |
| Sbjct: | 527 | AAATGCAAATGTTTGGAAATACTTCACAGACCTTTTTGACTACCTTCCTCTCACTGCCTT | 586 |
| Query: | 161 | GGTGGATGGGCAGATCTTCTGTCTACATGGTGGTCTTTCACCATCCATAGACACACTGGA | 220 |
| Sbjct: | 587 | GGTGGATGGGCAGATCTTCTGTCTACATGGTGGTCTTTCACCATCCATAGACACACTGGA | 646 |
| Query: | 221 | TCACATCCGAGCACTTGATCGCCTACAAGAAGTTCCTCATGAGGGTCCAATGTGTGACTT | 280 |
| Sbjct: | 647 | TCACATCCGAGCACTTGATCGCCTACAAGAAGTTCCTCATGAGGGTCCAATGTGTGACTT | 706 |
| Query: | 281 | GCTGTGGTCAGATCCAGATGACCGTGGTGGCTGGGGGATATCTCCTCGGGGAGCTGGTTA | 340 |
| Sbjct: | 707 | GCTGTGGTCAGATCCAGATGACCGTGGTGGCTGGGGGATATCTCCTCGGGGAGCTGGTTA | 766 |
| Query: | 341 | TACCTTT 347 | |
| Sbjct: | 767 | TACCTTT 773 | |

| | |
|---|---|
| 2. | With Rat type-2A protein phosphatase catalytic subunit mRNA |

(Accession No. M33114.1; SEQ ID NO: 9)
Identities = 306/307 (99%), Positives = 306/307 (99%)

| | | | |
|---|---|---|---|
| Query: | 41 | CGACAGCAGACAGATCACACAAGTTTATGGTTTCTACGATGAGTGTTTAAGGAAATACGG | 100 |
| Sbjct: | 467 | CGAGAGCAGACAGATCACACAAGTTTATGGTTTCTACGATGAGTGTTTAAGGAAATACGG | 526 |
| Query: | 101 | AAATGCAAATGTTTGGAAATACTTCACAGACCTTTTTGACTACCTTCCTCTCACTGCCTT | 160 |
| Sbjct: | 527 | AAATGCAAATGTTTGGAAATACTTCACAGACCTTTTTGACTACCTTCCTCTCACTGCCTT | 586 |
| Query: | 161 | GGTGGATGGGCAGATCTTCTGTCTACATGGTGGTCTTTCACCATCCATAGACACACTGGA | 220 |
| Sbjct: | 587 | GGTGGATGGGCAGATCTTCTGTCTACATGGTGGTCTTTCACCATCCATAGACACACTGGA | 646 |
| Query: | 221 | TCACATCCGAGCACTTGATCGCCTACAAGAAGTTCCTCATGAGGGTCCAATGTGTGACTT | 280 |
| Sbjct: | 647 | TCACATCCGAGCACTTGATCGCCTACAAGAAGTTCCTCATGAGGGTCCAATGTGTGACTT | 706 |
| Query: | 281 | GCTGTGGTCAGATCCAGATGACCGTGGTGGCTGGGGGATATCTCCTCGGGGAGCTGGTTA | 340 |
| Sbjct: | 707 | GCTGTGGTCAGATCCAGATGACCGTGGTGGCTGGGGGATATCTCCTCGGGGAGCTGGTTA | 766 |
| Query: | 341 | TACCTTT 347 | |
| Sbjct: | 767 | TACCTTT 773 | |

| | |
|---|---|
| 3. | With Rat mRNA for phosphatase 2A catalytic subunit isotype alpha |

(Accession No. X16043.1; SEQ ID NO: 10)
Identities = 306/307 (99%), Positives = 306/307 (99%)

| | | | |
|---|---|---|---|
| Query: | 41 | CGACAGCAGACAGATCACACAAGTTTATGGTTTCTACGATGAGTGTTTAAGGAAATACGG | 100 |
| Sbjct: | 526 | CGAGAGCAGACAGATCACACAAGTTTATGGTTTCTACGATGAGTGTTTAAGGAAATACGG | 585 |
| Query: | 101 | AAATGCAAATGTTTGGAAATACTTCACAGACCTTTTTGACTACCTTCCTCTCACTGCCTT | 160 |
| Sbjct: | 586 | AAATGCAAATGTTTGGAAATACTTCACAGACCTTTTTGACTACCTTCCTCTCACTGCCTT | 645 |
| Query: | 161 | GGTGGATGGGCAGATCTTCTGTCTACATGGTGGTCTTTCACCATCCATAGACACACTGGA | 220 |
| Sbjct: | 646 | GGTGGATGGGCAGATCTTCTGTCTACATGGTGGTCTTTCACCATCCATAGACACACTGGA | 705 |
| Query: | 221 | TCACATCCGAGCACTTGATCGCCTACAAGAAGTTCCTCATGAGGGTCCAATGTGTGACTT | 280 |
| Sbjct: | 706 | TCACATCCGAGCACTTGATCGCCTACAAGAAGTTCCTCATGAGGGTCCAATGTGTGACTT | 765 |
| Query: | 281 | GCTGTGGTCAGATCCAGATGACCGTGGTGGCTGGGGGATATCTCCTCGGGGAGCTGGTTA | 340 |
| Sbjct: | 766 | GCTGTGGTCAGATCCAGATGACCGTGGTGGCTGGGGGATATCTCCTCGGGGAGCTGGTTA | 825 |
| Query: | 341 | TACCTTT 347 | |
| Sbjct: | 826 | TACCTTT 832 | |

| | |
|---|---|
| 4. | With M. musculus mRNA for phosphatase 2A catalytic subunit, |

isotype alpha (Accession No. Z67745.1; SEQ ID NO: 11)
Identities = 299/307 (97%), Positives = 299/307 (97%)

| | | | |
|---|---|---|---|
| Query: | 41 | CGACAGCAGACAGATCACACAAGTTTATGGTTTCTACGATGAGTGTTTAAGGAAATACGG | 100 |
| Sbjct: | 354 | CGAGAGCAGACAGATCACACAGGTTTATGGGTTCTACGACGAGTGTTTAAGGAAATACGG | 413 |
| Query: | 101 | AAATGCAAATGTTTGGAAATACTTCACAGACCTTTTTGACTACCTTCCTCTCACTGCCTT | 160 |
| Sbjct: | 414 | AAATGCAAATGTTTGGAAATACTTCACAGACCTTTTTGACTATCTTCCTCTCACTGCCTT | 473 |
| Query: | 161 | GGTGGATGGGCAGATCTTCTGTCTACATGGTGGTCTTTCACCATCCATAGACACACTGGA | 220 |
| Sbjct: | 474 | GGTGGATGGGCAGATCTTCTGTCTACATGGTGGTCTGTCACCATCCATAGACACACTGGA | 533 |
| Query: | 221 | TCACATCCGAGCACTTGATCGCCTACAAGAAGTTCCTCATGAGGGTCCAATGTGTGACTT | 280 |
| Sbjct: | 534 | TCACATCCGAGCACTCGATCGCCTACAGGAAGTTCCTCATGAGGGTCCAATGTGTGACTT | 593 |
| Query: | 281 | GCTGTGGTCAGATCCAGATGACCGTGGTGGCTGGGGGATATCTCCTCGGGGAGCTGGTTA | 340 |
| Sbjct: | 594 | GCTGTGGTCAGATCCAGATGACCGTGGTGGCTGGGGGATATCTCCTCGGGGAGCTGGTTA | 653 |
| Query: | 341 | TACCTTT 347 | |
| Sbjct: | 654 | TACCTTT 660 | |

| | |
|---|---|
| 5. | With Mus musculus protein phosphatase type 2A catalytic subunit |

alpha isoform mRNA (Accession No. AF076192; SEQ ID NO: 12)
Identities = 298/307 (97%), Positives = 298/307 (97%)

| | | | |
|---|---|---|---|
| Query: | 41 | CGACAGCAGACAGATCACACAAGTTTATGGTTTCTACGATGAGTGTTTAAGGAAATACGG | 100 |
| Sbjct: | 549 | CGAGAGCAGACAGATCACACAGGTTTATGGGTTCTACGACGAGTGTTTAAGGAAATACGG | 608 |
| Query: | 101 | AAATGCAAATGTTTGGAAATACTTCACAGACCTTTTTGACTACCTTCCTCTCACTGCCTT | 160 |
| Sbjct: | 609 | AAATGCAAATGTTTGGAAATACTTCACAGACCTTTTTGACTATCTTCCTCTCACTGCCTT | 668 |
| Query: | 161 | GGTGGATGGGCAGATCTTCTGTCTACATGGTGGTCTTTCACCATCCATAGACACACTGGA | 220 |
| Sbjct: | 669 | GGTGGATGGGCAGATCTTCTGTCTACACGGTGGTCTGTCACCATCCATAGACACACTGGA | 728 |
| Query: | 221 | TCACATCCGAGCACTTGATCGCCTACAAGAAGTTCCTCATGAGGGTCCAATGTGTGACTT | 280 |
| Sbjct: | 729 | TCACATCCGAGCACTCGATCGCCTACAGGAAGTTCCTCATGAGGGTCCAATGTGTGACTT | 788 |
| Query: | 281 | GCTGTGGTCAGATCCAGATGACCGTGGTGGCTGGGGGATATCTCCTCGGGGAGCTGGTTA | 340 |
| Sbjct: | 789 | GCTGTGGTCAGATCCAGATGACCGTGGTGGCTGGGGGATATCTCCTCGGGGAGCTGGTTA | 848 |
| Query: | 341 | TACCTTT 347 | |
| Sbjct: | 849 | TACCTTT 855 | |

### Example 2

### Confirmation of PP2/NR2B Interaction by Co-Transformation

Specificity of the PP2A/NR2B interaction was demonstrated by isolating the library clone (the prey clone) and co-transforming it with the original NR2B bait construct in reporter strain AH109. The library clone was isolated by electroporating 1 μl of yeast DNA into E. coli cells. Standard DNA miniprep was performed on a culture grown up from resulting colonies. Co-transformation with original NR2B bait, and pGBKT7-Lamin as negative control were performed by standard small scale LiAc yeast transformation procedures. Several colonies per clone were streaked onto SC-trp-his plate and tested by re-streaking on control plate of SC-trp-leu and experimental plate of SC-trp-leu-his-ade+X-alpha-gal. Positive clones were identified which grew on both plates when transformed with bait, and only on the double drop out plate with Lamin.

These results demonstrated that the PP2A catalytic subunit fragment encoded by clone YH04A_CO₂ physically interacts with the C-terminal region of the NR2B subunit of NMDA-R.

### Example 3

### Cloning of Full Length cDNA Encoding PP2A Catalytic Subunit

In this experiment, the full length cDNA encoding the catalytic subunit (alpha isoform, Ppp2ca) of Rattus norvegicus protein phosphatase 2 (formerly 2A) was isolated. As described above, Clone YH04A_CO₂ showed 97-99% sequence identity to the various sequences encoding PP2A catalytic subunit. We designed primers, based on the published sequence of rat PP2A catalytic subunit alpha isoform (Accession No. NM_{—}017039.1; SEQ ID NO: 2) to perform RT PCR on rat adult brain oligo dT primed cDNA: 5′ primer atggacgagaagttgttcaccaag (SEQ ID NO:4) and 3′ primer ttacaggaagtagtctggggtacg (SEQ ID NO:5). The RT-PCR product was subsequently used as template for a second PCR in which the rat PP2A full length clone was tagged with HA. The primers used in the second PCR are: 5′ attgcggccgcaccatgtacccttacgacgttcctgattacgctagcctcgacgagaagttgttcaccaaggag (SEQ ID NO: 6) and 3′ ggcctcgagttacaggaagtagtctggggtacgacgag (SQ ID NO: 7).

PCR conditions are 94° C., 2 min, 94° C., 15 sec, 58° C., 30 sec, 72° C., 3 min, 35 cycles. The HA-tagged amplicon was cloned into PCR 4.0 TOPO vector. Positive clones carrying the PP2A C subunit (PP2A-C) cDNA were submitted to sequencing to confirm correct sequence. For expression analysis in HEK293 cells the PP2A-C cDNA was cloned into pRK5 expression vector.

### Example 4

### NMDA-R/PP2A Binding: Co-Immunoprecipitation

Co-immunoprecipitation experiments were performed to further confirm the physical interaction between NMDA-R and PP2A catalytic subunit.

HEK 293 cells were transfected with NR2B expression clone and PP2A expression clone. PP2A catalytic subunit (Accession No. NM_{—}017039.1; SEQ ID NO:2) was tagged with HA at the N-terminus. The co-immunoprecipitation was performed by using an anti-HA antibody for immunoprecipitation and the interaction with NR2B subunit was detected with anti-NR2B antibody subsequently. As a control NR2B was immunoprecipitated with anti-HA in the absence of the interactor HA-PP2A.

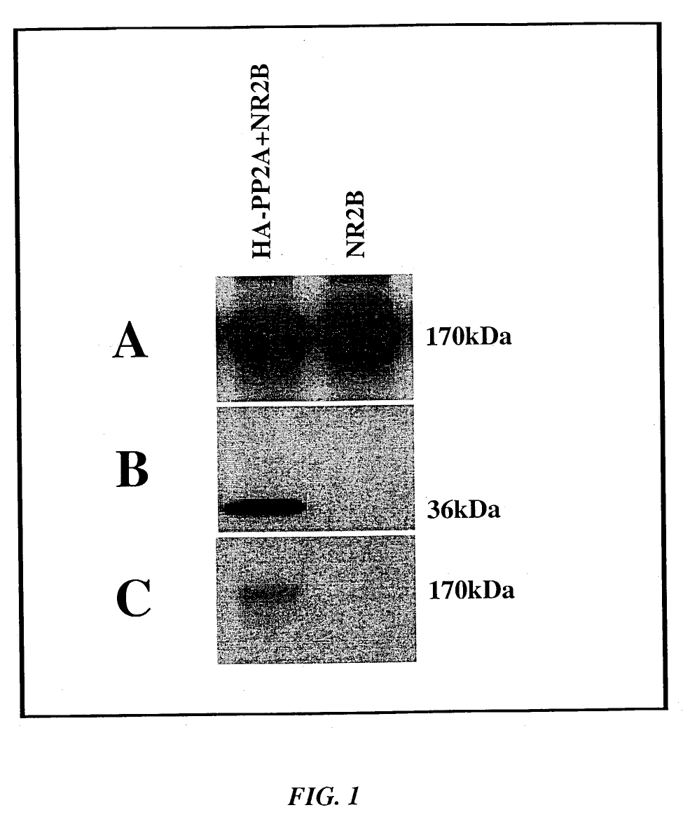

FIG. 1 exemplifies the results of such co-immunoprecipitation experiments. The right lane and left lane correspond respectively to results obtained from the PP2A/NR2B-expressing cell lysate and the NR2B-expressing (control) lysate. Panel A shows western blot of the lysates that were probed with the anti-NR2B antibody. Panel B shows western blot of the lysates that were probed with the anti-HA antibody. Panel C shows western blot of anti-HA immunoprecipitates of the lysates, probed with the anti-NR2B antibody. The results indicate that NR2B was co-precipitated with PP2A by anti-HA antibody from the lysate of HEK 293 cells expressing both PP2A and NR2B.

1. A method for identifying a modulator of N-methyl-D-aspartate receptor (NMDA-R) signaling activity, comprising detecting the ability of an agent to modulate the phosphatase activity of a serine/threonine protein phosphatase (PSTP) on a NMDA-R substrate or to modulate the binding of the PSTP to NMDA-R, thereby identifying the modulator, wherein the PSTP is capable of dephosphorylating NMDA-R.

2. The method of claim 1, wherein the PSTP is PP2A.

3. The method of claim 2, wherein the modulator is identified by detecting its ability to modulate the phosphatase activity of the PP2A.

4. The method of claim 1, wherein the modulator is identified by detecting its ability to modulate the binding of the PSTP to the NMDA-R.

5. A method for identifying an agent as a modulator of NMDA-R signaling, comprising:
(a) contacting
(i) the agent
(ii) PP2A; and
(iii) serine/threonine phosphorylated NMDA-R or a subunit thereof;
wherein either or both of (ii) and (iii) is substantially pure or recombinantly expressed;
(b) measuring the dephosphorylation activity of the PP2A on the NMDA-R or subunit;
(c) comparing the dephosphorylation activity in the presence of the agent with the dephosphorylation activity in the absence of the agent,
wherein a difference in the dephosphorylation activity identifies the agent as a modulator of NMDA-R signaling.

6. The method of claim 5, wherein the NMDA-R and the PP2A exist in a PP2A/NMDA-R-containing protein complex.

7. The method of claim 5, wherein the agent enhances the ability of the PP2A to dephosphorylate the NMDA-R.

8. The method of claim 5, wherein the agent inhibits the ability of the PP2A to dephosphorylate the NMDA-R.

9. The method of claim 5, wherein the agent modulates binding of the PP2A or the functional derivative thereof to the NMDA-R or the functional derivative thereof.

10. The method of claim 9, wherein the agent promotes or enhances the binding.

11. The method of claim 9, wherein the agent disrupts or inhibits the binding.

12. A method for identifying a nucleic acid molecule that modulates NMDA-R signaling, comprising:
(a) obtaining a cell culture coexpressing the NMDA-R and PP2A.
(b) introducing a nucleic acid molecule encoding a gene product into a portion of the cells; thereby producing cells comprising the nucleic acid molecule;
(c) culturing the cells in (b) under conditions in which the gene product is expressed;
(d) measuring PP2A dephosphorylation activity on the NMDA-R in the cells in (c) and comparing the dephosphorylation activity with that of control cells into which the nucleic acid molecule has not been introduced
wherein a difference in dephosphorylation activity identifies the nucleic acid molecule as a modulator of NMDA-R signaling.

13. A method for treating a disease mediated by abnormal NMDA-R-signaling, comprising administering a modulator of a PP2A activity, thereby modulating the level of seine/threonine phosphorylation of the NMDA-R.

14. The method of claim 13, wherein the modulator modulates the ability of PP2A to dephosphorylate NMDA-R.

15. The method of claim 13, wherein the modulator modulates the ability of PP2A to bind to NMDA-R.

16. The method of claim 13, wherein the modulator is a PP2A agonist, wherein the disease is selected from the group consisting of (i) ischemic stroke; (ii) head trauma or brain injury; (iii) Huntington's disease; (iv) spinocerebellar degeneration; (v) motor neuron diseases; (vi) epilepsy; (vii) neuropathic pain; (viii) chronic pain; and (ix) tolerance.

17. The method of claim 13, wherein the modulator is a PP2A antagonist, wherein the disease is selected from the group consisting of (i) schizophrenia; (ii) Alzheimer disease; (iii) dementia; (iv) psychosis; (v) depression; (vi) drug addiction; (vii) ethanol sensitivity; and (viii) attention disorder.


SEQUENCE LISTING (7 sequences)

SEQ ID NO: 1 - DNA, 347 bp, Rattus norvegicus
  misc_feature (1)...(347): n = A,T,C or G
atggcctgngagccccggggatccgaattcgcggccgcgtcgacagcagacagatcacacaagtttatggtttctacgatgagtgtttaaggaaatacggaaatgcaaatgtttggaaatacttcacagacctttttgactaccttcctctcactgccttggtggatgggcagatcttctgtctacatggtggtctttcaccatccatagacacactggatcacatccgagcacttgatcgcctacaagaagttcctcatgagggtccaatgtgtgacttgctgtggtcagatccagatgaccgtggtggctgggggatatctcctcggggagctggttataccttt

SEQ ID NO: 2 - DNA, 1804 bp, Rattus norvegicus
ctggggccgcaggaagcaccccggggagcggcggcggcgtgtgcgtgtggcccgggtgcgggcggcggcgcgggagcagcgcagagcggcagccggttcgggcgggcggcatcatggacgagaagttgttcaccaaggagctggaccagtggatcgagcagctgaacgagtgcaagcagctctccgagtcccaggtcaagagcctctgcgagaaggctaaagaaatcctgacaaaagaatctaatgttcaggaggttcgatgtccagtcactgtgtgtggagatgtgcatgggcaatttcatgacctcatggaactctttagaattggtggtaaatcaccagatacaaattacttgtttatgggagactatgtggacagaggatattactcagttgaaacagttacactgcttgtagctcttaaggttcgttaccgagagcgtatcaccatactccgagggaatcacgagagcagacagatcacacaagtttatggtttctacgatgagtgtttaaggaaatacggaaatgcaaatgtttggaaatacttcacagacctttttgactaccttcctctcactgccttggtggatgggcagatcttctgtctacatggtggtctttcaccatccatagacacactggatcacatccgagcacttgatcgcctacaagaagttcctcatgagggtccaatgtgtgacttgctgtggtcagatccagatgaccgtggtggctgggggatatctcctcggggagctggttatacctttggccaagatatttctgagacatttaatcatgccaatggcctcacgttggtgtccagagctcaccagctggtgatggagggatataactggtgccatgaccggaatgtagtaacaattttcagtgctccaaactattgctatcgttgtggtaaccaagctgcaatcatggaacttgatgacactcttaagtattctttcttgcagttcgatccagcacctcgtagaggcgagccacatgtcactcgtcgtaccccagactacttcctgtaatgaaagtttaaccttgtacagtattgccatgaacaccgtctgttgacctaatggaatcgggaagagcagcagtaactccaaagtgtcagaaatagttaacattcaaacttgtttccacacggaccaaaagatgtgccatataaaatacaaagcctcttgtcatcaacagccgtgaccactttagaatgaaccagttcattgcatgctgacgcgacattgttggtcaagaatccagtttctggcatagcgctatttgtagttacttttgctttcttgagagactgcagatctaggatgtaacattaacacctgtgagtccagttgacttccacttagctgtagcttactcagcatgactgtagatgaggatagcaaacaatcattggagcttaatgaacatttttaaatgagtaccaaggcctcccctcttgttgtgttctttcagggatactattaatttaattgtatgatttctctgcactcagtttctcccttctcaaatctcggccccgcgttgttctttgttactgtcagaaaacctggtgagttgttttgaacagaactgtctccctcctgtaagatgatgtactgcacaagtcaccgcagtgttttcataataaacttgagaactgagaaagtcaggtttgaattgtatcagtgggcacgactggtgctgtttattaaacaagataaatctattgatcaatttcagaatttgtagaattccaggtaaagaaaaataaagatcaaggccactatat

SEQ ID NO: 3 - protein, 309 aa, Rattus norvegicus
MDEKLFTKELDQWIEQLNECKQLSESQVKSLCEKAKEILTKESNVQEVRCPVTVCGDVHGQFHDLMELFRIGGKSPDTNYLFMGDYVDRGYYSVETVTLLVALKVRYRERITILRGNHESRQITQVYGFYDECLRKYGNANVWKYFTDLFDYLPLTALVDGQIFCLHGGLSPSIDTLDHIRALDRLQEVPHEGPMCDLLWSDPDDRGGWGISPRGAGYTFGQDISETFNHANGLTLVSRAHQLVMEGYNWCHDRNVVTIFSAPNYCYRCGNQAAIMELDDTLKYSFLQFDPAPRRGEPHVTRRTPDYFL

SEQ ID NO: 4 - DNA, 24 bp, Artificial Sequence
  PCR 5′ primer
atggacgagaagttgttcaccaag

SEQ ID NO: 5 - DNA, 24 bp, Artificial Sequence
  PCR 3′ primer
ttacaggaagtagtctggggtacg

SEQ ID NO: 6 - DNA, 74 bp, Artificial Sequence
  PCR 5′ primer
attgcggccgcaccatgtacccttacgacgttcctgattacgctagcctcgacgagaagttgttcaccaaggag

SEQ ID NO: 7 - DNA, 38 bp, Artificial Sequence
  PCR 3′ primer
ggcctcgagttacaggaagtagtctggggtacgacgag